(19) United States
(12) Patent Application Publication
(10) Pub. No.: US 2025/0127421 A1
(43) Pub. Date: Apr. 24, 2025

(54) MOTION STATE MONITORING SYSTEM, METHOD FOR CONTROLLING THE SAME, AND NON-TRANSITORY COMPUTER READABLE MEDIUM STORING CONTROL PROGRAM

(71) Applicant: TOYOTA JIDOSHA KABUSHIKI KAISHA, Toyota-shi (JP)

(72) Inventors: Makoto KOBAYASHI, Nisshin-shi (JP); Yoshiaki KATO, Miyoshi-shi (JP)

(73) Assignee: TOYOTA JIDOSHA KABUSHIKI KAISHA, Toyota-shi (JP)

(21) Appl. No.: 18/819,901

(22) Filed: Aug. 29, 2024

(30) Foreign Application Priority Data
Oct. 24, 2023 (JP) 2023-182854

(51) Int. Cl. A61B 5/11; G06F 3/04817; G06F 3/0486

(52) U.S. Cl. CPC A61B 5/11; G06F 3/04817; G06F 3/0486

(57) ABSTRACT

A motion state monitoring system according to the present disclosure is a motion state monitoring system configured to monitor a motion of a subject based on a result of detection by each of a plurality of sensors attached to a plurality of respective body parts of a body of the subject, the motion state monitoring system including: a display control unit configured to display, on one screen, a measurement result icon indicating a result of measurement of a motion to be monitored and a display setting area for setting an area that displays the result of measurement, the result of measurement being acquirable from the sensor attached to the body part of the body of the subject; and an acceptance unit configured to accept a setting operation for the display setting area for setting an area that displays the result of measurement for the measurement result icon.

### CROSS REFERENCE TO RELATED APPLICATIONS

This application is based upon and claims the benefit of priority from Japanese patent application No. 2023-182854, filed on Oct. 24, 2023, the disclosure of which is incorporated herein in its entirety by reference.

### BACKGROUND

The present disclosure relates to a motion state monitoring system, a method for controlling the same, and a control program.

Patent Literature 1 discloses a motion state monitoring system that monitors a motion state of a subject based on a result of detection by a plurality of sensors attached to a plurality of respective body parts of a body of the subject.
Patent Literature 1: Japanese Unexamined Patent Application Publication No. 2022-34449

### SUMMARY

The motion state monitoring system that monitors a motion state of a subject as disclosed in the related art is required to improve its usability for users with regard to a setting for displaying results of monitoring on a monitor.

The present disclosure has been made in view of the aforementioned circumstances and an object thereof is to provide a motion state monitoring system, a method for controlling the same, and a control program the usability of which can be improved.

A motion state monitoring system according to the present disclosure is a motion state monitoring system configured to monitor a motion of a subject based on a result of detection by each of a plurality of sensors attached to a plurality of respective body parts of a body of the subject, the motion state monitoring system including: a display control unit configured to display, on one screen, a measurement result icon indicating a result of measurement of a motion to be monitored and a display setting area for setting an area that displays the result of measurement, the result of measurement being acquirable from the sensor attached to the body part of the body of the subject; and an acceptance unit configured to accept a setting operation for the display setting area for setting an area that displays the result of measurement for the measurement result icon. In the above-described motion state monitoring system, a user simply moves the measurement result icon displayed on the screen to the display setting area, whereby the result of measurement which the user wants to display on the screen is displayed on the screen, for example, in the form of a graph. That is, the usability of the above-described motion state monitoring system can be improved.

Further, the above-described motion state monitoring system may perform calculation processing by using a trained model generated by machine learning using a result of the past detection of the sensor. In the above-described motion state monitoring system, by performing calculation processing using a trained model, it is possible to more accurately calculate whether or not a motion state of a motion to be monitored of a subject is satisfactory.

The display control unit may display, on one screen, a plurality of measurement result icons respectively indicating a plurality of results of measurement of a plurality of motions to be monitored and a plurality of display setting areas for setting a plurality of areas each of which displays a selected one of the plurality of results of measurement, the results of measurement being acquirable from the plurality of sensors attached to the body parts of the body of the subject.

The acceptance unit may accept a setting operation for the common display setting area for setting an area that displays the results of measurement for two or more of the plurality of measurement result icons.

The acceptance unit may accept the setting operation by an operation for dragging and dropping the measurement result icon onto the display setting area, the operation being input through an operation terminal.

The display control unit may display, on the screen, the measurement result icon indicating the result of measurement which is determined to be displayed in the display setting area.

The acceptance unit may accept a change operation for changing an area that displays the result of measurement by an operation for dragging and dropping the measurement result icon displayed in one of the display setting areas onto another display setting area, the operation being input through an operation terminal.

The plurality of results of measurement may include angles of joints of the body of the subject based on the results of detection by the plurality of sensors or the angles of joints in any coordinate system based on one of the result of detection by the plurality of sensors.

The motion state monitoring system according to the present disclosure may further include the plurality of sensors.

A method for controlling a motion state monitoring system according to the present disclosure is a method for controlling a motion state monitoring system configured to monitor a motion of a subject based on a result of detection by each of a plurality of sensors attached to a plurality of respective body parts of a body of the subject, the method including: displaying, on one screen, a measurement result icon indicating a result of measurement of a motion to be monitored and a display setting area for setting an area that displays the result of measurement, the result of measurement being acquirable from the sensor attached to the body part of the body of the subject; and accepting a setting operation for the display setting area for setting an area that displays the result of measurement for the measurement result icon. In above-described method for controlling a motion state monitoring system, a user simply moves the measurement result icon displayed on the screen to the display setting area, whereby the result of measurement which the user wants to display on the screen is displayed on the screen, for example, in the form of a graph. That is, the usability of the above-described method for controlling a motion state monitoring system can be improved.

A control program according to the present disclosure is a control program for causing a computer to execute control processing in a motion state monitoring system configured to monitor a motion of a subject based on a result of detection by each of a plurality of sensors attached to a plurality of respective body parts of a body of the subject, the control processing including: displaying, on one screen, a measurement result icon indicating a result of measurement of a motion to be monitored and a display setting area for setting an area that displays the result of measurement, the result of measurement being acquirable from the sensor attached to the body part of the body of the subject; and accepting a setting operation for the display setting area for setting an area that displays the result of measurement for the measurement result icon. In above-described control program, a user simply moves the measurement result icon displayed on the screen to the display setting area, whereby the result of measurement which the user wants to display on the screen is displayed on the screen, for example, in the form of a graph. That is, the usability of the above-described control program can be improved.

According to the present disclosure, it is possible to provide a motion state monitoring system, a method for controlling the same, and a control program the usability of which can be improved.

The above and other objects, features and advantages of the present disclosure will become more fully understood from the detailed description given hereinbelow and the accompanying drawings.

### BRIEF DESCRIPTION OF DRAWINGS

FIG. 1 is a block diagram showing an example of a configuration of a motion state monitoring system according to a first embodiment;

FIG. 2 is a diagram showing an example of body parts of a subject to which measuring instruments are to be attached;

FIG. 3 is a diagram showing an example of a configuration of the measuring instrument provided in the motion state monitoring system according to the first embodiment;

FIG. 4 is a diagram showing an example of how to attach the measuring instruments shown in FIG. 3 to a subject;

FIG. 5 is a flowchart showing operations performed by a motion state monitoring apparatus provided in the motion state monitoring system according to the first embodiment;

FIG. 6 is a diagram showing an example of a content displayed on a monitor;

FIG. 7 is a diagram showing an example of a content displayed on a monitor; and

FIG. 8 is a diagram showing an example of a content displayed on a monitor.

### DESCRIPTION OF EMBODIMENTS

The present disclosure will be described hereinafter through embodiments of the present disclosure. However, the disclosure according to the claims is not limited to the following embodiments. Further, all the components/structures described in the embodiments are not necessarily essential as means for solving the problem. For the clarification of the description, the following descriptions and the drawings are partially omitted and simplified as appropriate. The same elements are denoted by the same reference numerals or symbols throughout the drawings, and redundant descriptions are omitted as necessary.

### First Embodiment

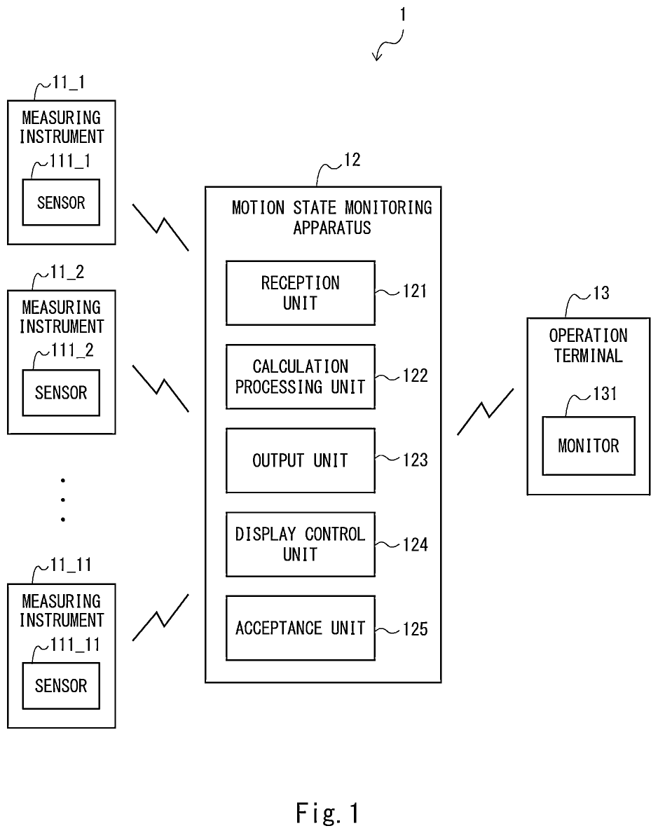

FIG. 1 is a block diagram showing a configuration example of a motion state monitoring system 1 according to a first embodiment. The motion state monitoring system 1 is a system that monitors a motion state of a subject. By a result of the monitoring, for example, a user, such as an assistant, can provide support for making the motion of the subject close to a desired motion. The details thereof will be described below.

As shown in FIG. 1, the motion state monitoring system 1 includes a plurality of measuring instruments 11, a motion state monitoring apparatus 12, and an operation terminal 13. The motion state monitoring apparatus 12 itself can be referred to as a motion state monitoring system. The motion state monitoring apparatus 12, the plurality of measuring instruments 11, and the operation terminal 13 are configured so that they can communicate with each other through a wired or wireless network. Note that, in this embodiment, an example of a case in which 11 of the measuring instruments 11 are provided will be described. In the following description, the 11 measuring instruments 11 are also referred to as measuring instruments 11_1 to 11_11, respectively, in order to distinguish them from each other.

The operation terminal 13 is a terminal capable of performing communication owned by a user or temporarily assigned to a user, such as a Personal Computer (PC) terminal, a mobile terminal such as a smartphone or a tablet terminal, or a dedicated communication terminal prepared for the motion state monitoring system 1. Note that, in this embodiment, a description will be given of a case in which the operation terminal 13 and the motion state monitoring apparatus 12 are separately provided. However, the present disclosure is not limited thereto; for example, the operation terminal 13 and the motion state monitoring apparatus 12 may be integrally formed.

For example, a user operates a monitor 131 of the operation terminal 13 by touching it with a stylus pen or a finger, or operates a mouse, a keyboard, or the like of the operation terminal 13, thereby inputting information about a subject, a result of monitoring which the user wants to display on the monitor 131, and the like to the operation terminal 13. The operation terminal 13 receives the above information and transmits it to the motion state monitoring apparatus 12 through the network. When the monitor 131 receives an operation performed by a user, the monitor 131 displays a screen for inputting information about a subject or a screen for selecting a result of monitoring which the user wants to display on the monitor 131, while when the motion state monitoring apparatus 12 has finished monitoring the motion state of the subject, the monitor 131 displays a result of monitoring received from the motion state monitoring apparatus 12.

The measuring instruments 11_1 to 11_11 are respectively attached to body parts 20_1 to 20_11 of a subject P from which motions are to be detected among various body parts of the body of the subject P, and detect the motions of the respective body parts 20_1 to 20_11 by using motion sensors (hereinafter simply referred to as sensors) 111_1 to 111_11 composed of gyro sensors, acceleration sensors, and the like. Note that the measuring instruments 11_1 to 11_11 are made to correspond to the respective body parts 20_1 to 20_11 by pairing processing performed with the motion state monitoring apparatus 12.

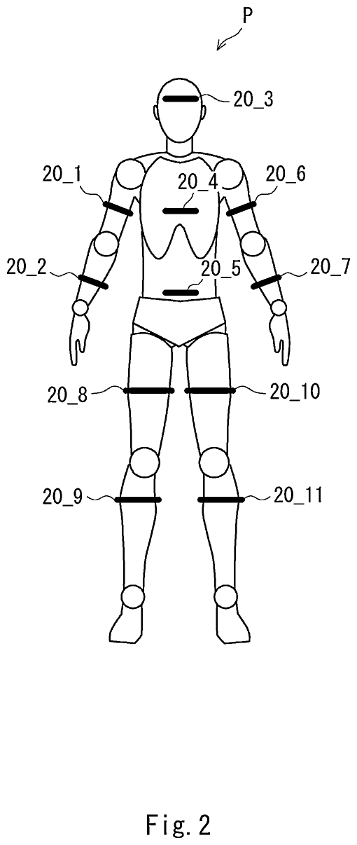

FIG. 2 is a diagram showing an example of body parts of the subject P to which the measuring instruments 11_1 to 11_11 are to be attached. In the example shown in FIG. 2, the body parts 20_1 to 20_11 to which the respective measuring instruments 11_1 to 11_11 are to be attached are a right upper arm, a right forearm, a head, a chest (a trunk), a waist (a pelvis), a left upper arm, a left forearm, a right thigh, a right lower leg, a left thigh, and a left lower leg, respectively. Note that it is not necessary to attach all of the measuring instruments 11_1 to 11_11 to the body of the subject P. It is only necessary to attach, among the measuring instruments 11_1 to 11_11, only the measuring instrument(s) required to measure a motion to be monitored (including the movement of the body part) which a user wants to monitor to the body of the subject P.

### (Examples of Configurations of Measuring Instruments 11_1 to 11_11)

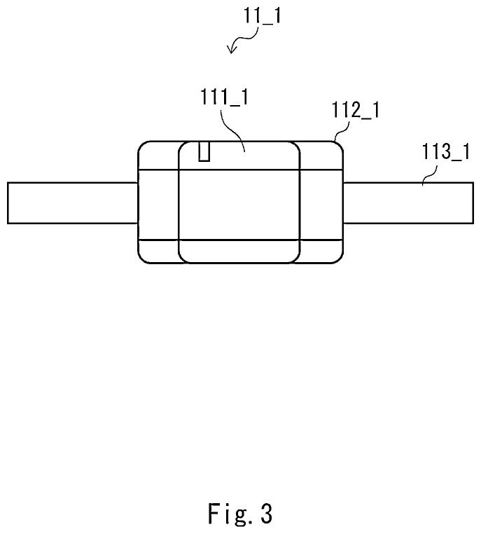

FIG. 3 is a diagram showing an example of a configuration of the measuring instrument 11_1. Note that the configuration of each of the measuring instruments 11_2 to 11_11 is similar to that of the measuring instrument 11_1, and thus the descriptions thereof will be omitted.

As shown in FIG. 3, the measuring instrument 11_1 includes the sensor 111_1, an attachment pad 112_1, and a belt 113_1. The belt 113_1 is configured so that it can be wound around the body part of the subject P from which a motion is to be detected. The sensor 111_1 is integrated with, for example, the attachment pad 112_1. The attachment pad 112_1 with which the sensor 111_1 is integrated is configured so that it can be attached to or detached from the belt 113_1.

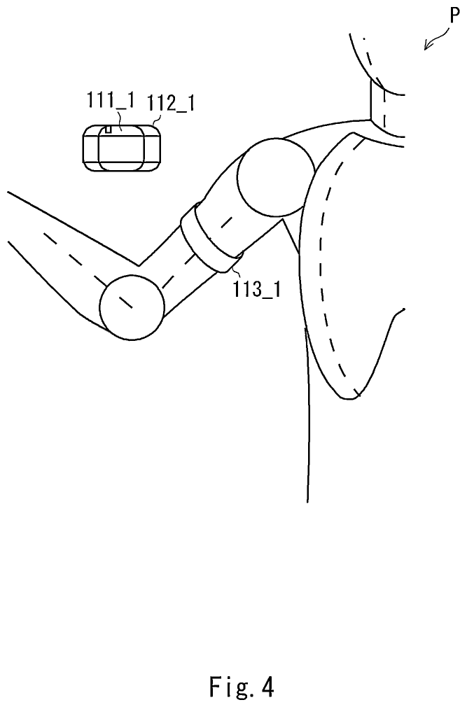

FIG. 4 is a diagram showing an example of how to attach the measuring instrument 11_1 to the subject P. In the example shown in FIG. 4, the belt 113_1 is wound around the right upper arm which is one of the body parts of the subject P from which motions are to be detected. The sensor 111_1 is attached to the belt 113_1 with the attachment pad 112_1 interposed therebetween after pairing, a calibration, and the like have been completed.

In FIG. 1, the motion state monitoring apparatus 12 outputs a result of a calculation indicating a motion state of the subject P based on results (sensing values) of detection by the sensors 111_1 to 111_11.

Specifically, the motion state monitoring apparatus 12 includes a reception unit 121, a calculation processing unit 122, an output unit 123, a display control unit 124, and an acceptance unit 125.

The reception unit 121 receives results of detection by the sensor 111_1 to 111_11 and receives information input to the operation terminal 13 by a user.

The calculation processing unit 122 performs calculation processing based on a result of detection by each of the sensors 111_1 to 111_11 and generates a result of the calculation indicating a motion state of a motion to be monitored of the subject P. Examples of the motion to be monitored include motions such as bending and stretching of the right shoulder, adduction and abduction of the right shoulder, internal and external rotation of the right shoulder, bending and stretching of the right elbow, pronation and supination of the right forearm, bending and stretching of the head, rotation of the head, bending and stretching of the chest and the waist, rotation of the chest and the waist, lateral bending of the chest and the waist, bending and stretching of the left shoulder, adduction and abduction of the left shoulder, internal and external rotation of the left shoulder, bending and stretching of the left elbow, and pronation and supination of the left forearm. Further, the motion to be monitored includes movement of the body part to which a sensor is attached. The motion to be monitored also includes, for example, an angle of the joint of the body of the subject P to be measured based on results of the detection by a plurality of sensors and an angle of the joint in any coordinate system to be measured based on a result of the detection by one of the sensors. In the following description, generation of a result of calculation indicating a motion state of a motion to be monitored is also referred to as measurement of a motion to be monitored.

For example, the calculation processing unit 122 performs calculation processing based on a result of detection by each of the sensor 111_1 attached to the right upper arm (the body part 20_1) and the sensor 111_2 attached to the right forearm (the body part 20_2) of the subject P among the sensors 111_1 to 111_11, and generates a result of the calculation indicating a motion state of the bending and stretching motion of the right elbow of the subject P.

Alternatively, the calculation processing unit 122 performs calculation processing based on a result of detection by each of the sensor 111_5 attached to the waist (the body part 20_5) and the sensor 111_8 attached to the right thigh (the body part 20_8) of the subject P among the sensors 111_1 to 111_11, and generates a result of the calculation indicating a motion state of the lateral bending motion of the right side of the waist of the subject P.

Note that the calculation processing unit 122 may perform calculation processing by using a trained model generated by machine learning using a result of the past detection of the sensor. By performing calculation processing using the trained model, the calculation processing unit 122 can more accurately calculate whether or not a motion state of a motion to be monitored of the subject P is satisfactory.

The output unit 123 outputs a result of the calculation by the calculation processing unit 122. The information (the result of the calculation) output from the output unit 123 is transferred to the operation terminal 13 through the network, visualized in the form of a graph or the like, and displayed on the monitor 131 of the operation terminal 13. As a result, a user can know the motion state of the motion to be monitored of the subject P, and can, for example, use it for assisting the subject P.

In order to make a user select a result of the measurement of the motion to be monitored to be displayed on the monitor 131, the display control unit 124 displays an icon display area S1 and display setting areas A1 to A3 on a screen of the monitor 131. Note that the number of display setting areas is not limited to three, and may be one or more. Further, the display control unit 124 displays a measurement result icon indicating a result of measurement of the motion to be monitored that can be displayed on the monitor 131 in the icon display area S1. In other words, the display control unit 124 displays a measurement result icon indicating a result of measurement of the motion to be monitored that can be acquired from the sensor attached to the subject P. A user can select, from among the measurement result icons displayed in the icon display area S1, a measurement result icon corresponding to a result of the measurement of the motion to be monitored which the user wants to display on the monitor 131.

The acceptance unit 125 accepts a setting operation performed by a user for one of the display setting areas A1 to A3 for setting areas that display the details of the results of measurement for the measurement result icons displayed in the icon display area S1. For example, a user moves the measurement result icon displayed in the icon display area S1 to the display setting area A1. Specifically, a user drags and drops the measurement result icon displayed in the icon display area S1 onto the display setting area A1 by a mouse operation, a touch operation, or the like. By doing so, the acceptance unit 125 accepts a setting operation performed by a user for the display setting area A1 for setting an area that displays the details of the result of measurement for the measurement result icon. As a result, the display setting area A1 displays the details of the selected result of measurement (e.g., a graphed result of measurement).

Note that, in this embodiment, although a description has been given of an example of a case in which the acceptance unit 125 accepts a setting operation performed by a user for the display setting area A1 for setting an area that displays the details of a result of measurement for one measurement result icon displayed in the icon display area S1, the present disclosure is not limited to this case. The acceptance unit 125 may further accept a setting operation performed by a user for one of the display setting areas A2 and A3 for setting areas that display the details of a result of measurement for another measurement result icon displayed in the icon display area S1. In this case, for example, a user moves one measurement result icon displayed in the icon display area S1 to the display setting area A1 and moves another measurement result icon displayed in the icon display area S1 to one of the display setting areas A2 and A3. By doing so, the acceptance unit 125 accepts a setting operation performed by a user for the display setting area A1 for setting an area that displays the details of a result of measurement for one measurement result icon and a setting operation performed by the user to one of the display setting areas A2 and A3 for setting areas that display the details of a result of measurement for another measurement result icon. As a result, the details of one selected result of measurement are displayed in the display setting area A1 and the details of another selected result of measurement are displayed in one of the display setting areas A2 and A3.

Further, in this embodiment, although a description has been given of an example of a case in which the acceptance unit 125 accepts a setting operation performed by a user for the display setting area A1 for setting an area that displays the details of a result of measurement for one measurement result icon displayed in the icon display area S1, the present disclosure is not limited to this case. The acceptance unit 125 may accept a setting operation performed by a user for the display setting area A1 for setting an area that displays the details of a result of measurement for another measurement result icon displayed in the icon display area S1. In this case, for example, a user moves two measurement result icons displayed in the icon display area S1 to the display setting area A1. By doing so, the acceptance unit 125 accepts a setting operation performed by a user for the common display setting area A1 for setting an area that displays the details of a result of measurement for the two measurement result icons. As a result, the display setting area A1 displays the details of the two selected results of measurement. For example, two graphed results of measurement are displayed in the display setting area A1 while they are superimposed on each other.

Further, the acceptance unit 125 may accept a change operation for changing an area that displays the result of measurement corresponding to the measurement result icon from the display setting area A1 to one of the display setting areas A2 and A3 in response to, for example, a user moving (dragging and dropping) the measurement result icon from the display setting area A1 onto one of the display setting areas A2 and A3. Further, the measurement result icon set in the display setting area A1 may be set in one or both of the display setting areas A2 and A3 at the same time.

### (Operation of Motion State Monitoring Apparatus 12)

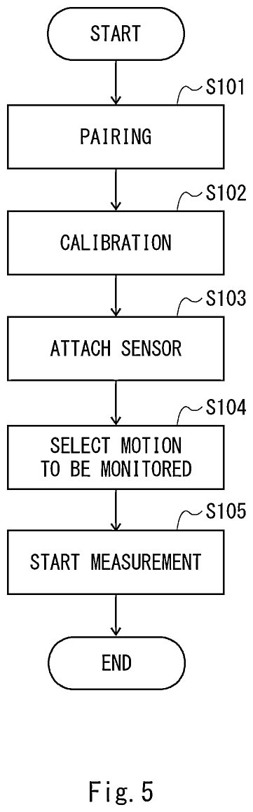

Next, operations performed by the motion state monitoring apparatus 12 will be described with reference to FIG. 5. FIG. 5 is a flowchart showing the operations performed by the motion state monitoring apparatus 12.

First, the motion state monitoring apparatus 12 performs pairing processing between the motion state monitoring apparatus 12 and each of the measuring instruments 11_1 to 11_11, thereby making the measuring instruments 11_1 to 11_11 correspond to the body parts 20_1 to 20_11 of the subject P (Step S101).

After that, the motion state monitoring apparatus 12 performs a calibration of the sensors 111_1 to 111_11 (Step S102). A calibration is, for example, processing for measuring an output value (an error component) of a sensor in a standstill state, the sensor being used to measure a motion to be monitored, and subtracting the error component from a measured value. It is assumed here that the output value of the sensor is stabilized after a predetermined period of time (about 20 seconds) has elapsed from when the sensor is brought to a standstill. In this case, in the calibration, it is desirable that the output value of the sensor after a predetermined period of time has elapsed from when the sensor is brought to a standstill be used as an error component. Therefore, in this example, the output value of the sensor after a predetermined period of time has elapsed from when a user has given an instruction to start the calibration after the sensor has been brought to a standstill is used as an error component. Further, “during the calibration” means a processing period of time until an error component is determined, and “completion of the calibration” means that the output value (the error component) of the sensor in a standstill state has been determined.

During the calibration, the monitor 131 displays, for example, the information that “Calibration is in progress. Place the sensor on the desk and do not move it”. Upon completion of the calibration, the monitor 131 displays, for example, the information that “Calibration has been completed. Attach the sensor”. Note that the information indicating that the calibration is in progress or that the calibration has been completed is not limited to being given by displaying it on the monitor 131, and may instead be given by other notification methods such as by voice. Further, the order in which the calibration processing and the pairing processing are performed may be reversed.

After the calibration has been completed, the sensor is attached to the subject P (Step S103). In this example, among the sensors 111_1 to 111_11, the sensors 111_1, 111_2, 111_5, and 111_8 are attached to the right upper arm (the body part 20_1), the right forearm (the body part 20_2), the waist (the body part 20_5), and the right thigh (the body part 20_8) of the subject P, respectively.

After that, the motion state monitoring apparatus 12 measures a motion to be monitored that can be measured using the sensors attached to the subject P among a plurality of motions to be monitored (Step S104).

After that, the motion state monitoring apparatus 12 outputs a result of the measurement (a result of the calculation indicating a motion state of the motion to be monitored of the subject P) (Step S105).

Specifically, the motion state monitoring apparatus 12 first displays the icon display area S1 and the display setting areas A1 to A3 on the screen of the monitor 131. Note that the number of display setting areas is not limited to three, and may be one or more. Further, the motion state monitoring apparatus 12 displays a measurement result icon indicating a result of measurement of the motion to be monitored that can be displayed on the monitor 131 in the icon display area S1. In other words, the motion state monitoring apparatus 12 displays a measurement result icon indicating a result of measurement of the motion to be monitored that can be acquired from the sensor attached to the subject P in the icon display area S1. A user can select, from among the measurement result icons displayed in the icon display area S1, a measurement result icon corresponding to a result of measurement of the motion to be monitored which the user wants to display on the monitor 131.

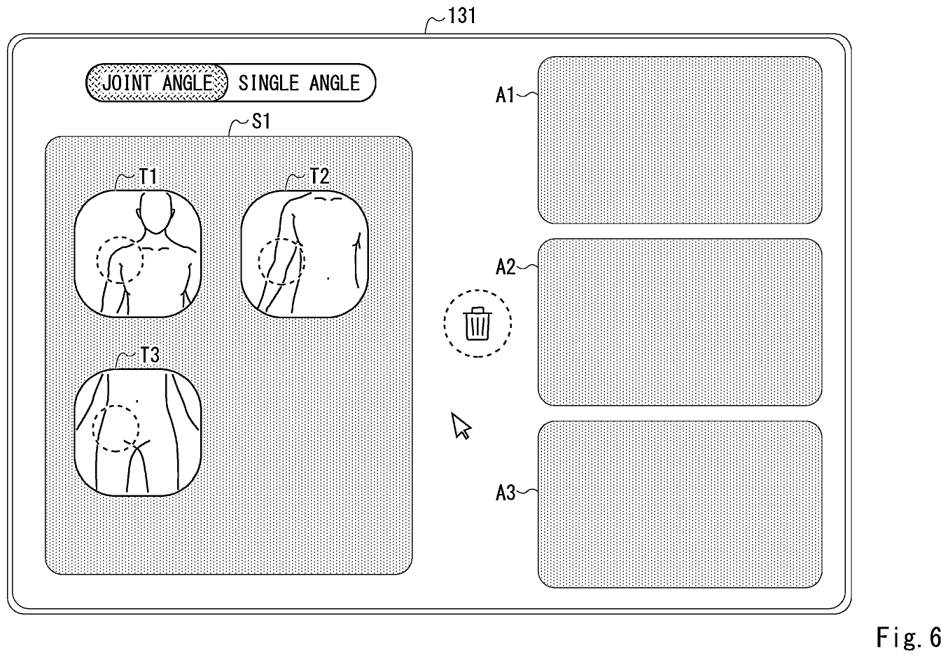

In the example shown in FIG. 6, the icon display area S1 is displayed on the left side of the screen of the monitor 131, while the display setting areas A1 to A3 are displayed on the right side of the screen of the monitor 131. Further, the icon display area S1 displays a measurement result icon T1 indicating a result of measurement of the “adduction and abduction motion of the right shoulder” that can be acquired from the sensor 111_1, a measurement result icon T2 indicating a result of measurement of the “bending and stretching motion of the right elbow” that can be acquired from the sensors 111_1 and 111_2, and a measurement result icon T3 indicating a result of measurement of the “lateral bending motion of the right side of the waist” that can be acquired from the sensors 111_5 and 111_8. That is, in the example shown in FIG. 6, a user can select, from among the three measurement result icons T1 to T3 displayed in the icon display area S1, a measurement result icon corresponding to a result of the measurement of the motion to be monitored which the user wants to display on the monitor 131.

After that, the motion state monitoring apparatus 12 accepts a setting operation performed by a user for one of the display setting areas A1 to A3 for setting areas that display the details of the results of measurement for the measurement result icons T1 to T3 displayed in the icon display area S1.

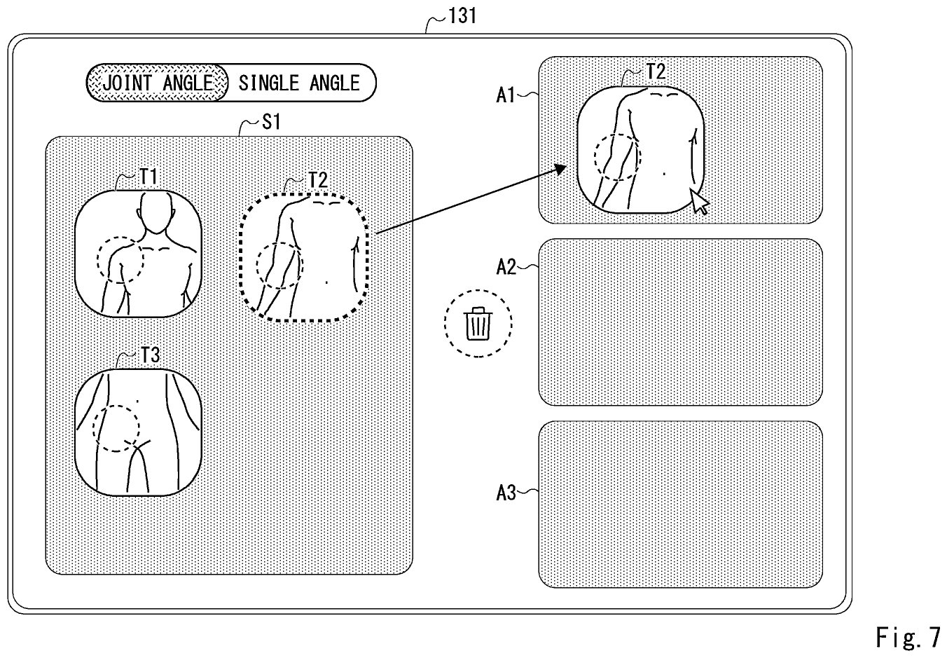

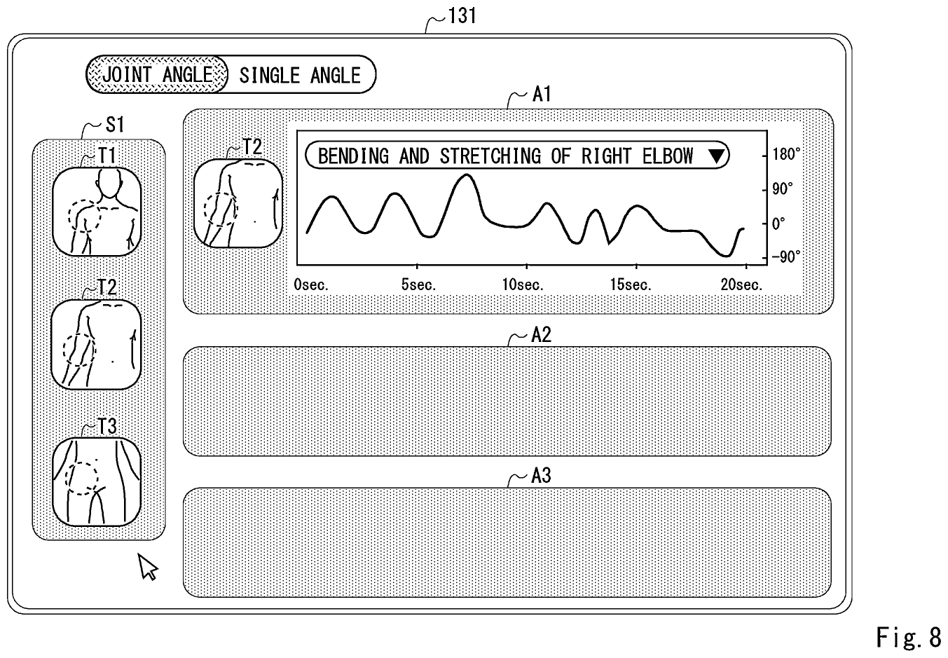

In the example shown in FIG. 7, a user drags and drops the measurement result icon T1 among the measurement result icons T1 to T3 displayed in the icon display area S1 onto the display setting area A1 by a mouse operation, a touch operation, or the like. By doing so, the motion state monitoring apparatus 12 accepts a setting operation performed by a user for the display setting area A1 for setting an area that displays the details of the result of measurement for the measurement result icon T1. As a result, as shown in the example of FIG. 8, the display setting area A1 displays the details of the result of measurement (e.g., a graphed result of measurement) corresponding to the measurement result icon T1. Note that, even when the measurement result icon T1 is displayed in the display setting area A1 by a drag-and-drop operation, it is still also displayed in the icon display area S1. However, the measurement result icon T1 may be deleted from the icon display area S1 in response to the display of the measurement result icon T1 in the display setting area A1 by a drag-and-drop operation.

As described above, in the motion state monitoring system 1 according to this embodiment, a user simply moves the measurement result icon displayed on the screen of the monitor 131 to the display setting area, whereby the result of measurement of the motion to be monitored which the user wants to display on the screen is displayed on the screen, for example, in the form of a graph. That is, the usability of the motion state monitoring system according to this embodiment can be improved.

In this embodiment, although an example of a case in which a calibration is performed on all the sensors 111_1 to 111_11 has been described, the present disclosure is not limited to this case. For example, among the sensors 111_1 to 111_11, a calibration may be performed on only the sensor(s) attached to the body of the subject P.

Further, in the present disclosure, it is possible to implement some or all of the processes performed in the motion state monitoring system 1 by causing a Central Processing Unit (CPU) to execute a computer program.

The above-described program includes instructions (or software codes) that, when loaded into a computer, cause the computer to perform one or more of the functions described in the example embodiments. The program may be stored in a non-transitory computer readable medium or a tangible storage medium. By way of example, and not a limitation, non-transitory computer readable media or tangible storage media can include a Random-Access Memory (RAM), a Read-Only Memory (ROM), a flash memory, a Solid-State Drive (SSD) or other types of memory technologies, a CD-ROM, a Digital Versatile Disc (DVD), a Blu-ray (Registered Trademark) disc or other types of optical disc storage, a magnetic cassette, a magnetic tape, and a magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A motion state monitoring system configured to monitor a motion of a subject based on a result of detection by each of a plurality of sensors attached to a plurality of respective body parts of a body of the subject, the motion state monitoring system comprising:
a display control unit configured to display, on one screen, a measurement result icon indicating a result of measurement of a motion to be monitored and a display setting area for setting an area that displays the result of measurement, the result of measurement being acquirable from the sensor attached to the body part of the body of the subject; and
an acceptance unit configured to accept a setting operation for the display setting area for setting an area that displays the result of measurement for the measurement result icon.

2. The motion state monitoring system according to claim 1, wherein the display control unit displays, on one screen, a plurality of measurement result icons respectively indicating a plurality of results of measurement of a plurality of motions to be monitored and a plurality of display setting areas for setting a plurality of areas each of which displays a selected one of the plurality of results of measurement, the results of measurement being acquirable from the plurality of sensors attached to the body parts of the body of the subject.

3. The motion state monitoring system according to claim 2, wherein the acceptance unit accepts a setting operation for the common display setting area for setting an area that displays the results of measurement for two or more of the plurality of measurement result icons.

4. The motion state monitoring system according to claim 2, wherein the acceptance unit accepts the setting operation by an operation for dragging and dropping the measurement result icon onto the display setting area, the operation being input through an operation terminal.

5. The motion state monitoring system according to claim 4, wherein the display control unit displays, on the screen, the measurement result icon indicating the result of measurement which is determined to be displayed in the display setting area.

6. The motion state monitoring system according to claim 5, wherein the acceptance unit accepts a change operation for changing an area that displays the result of measurement by an operation for dragging and dropping the measurement result icon displayed in one of the display setting areas onto another display setting area, the operation being input through an operation terminal.

7. The motion state monitoring system according to claim 1, wherein the plurality of results of measurement include angles of joints of the body of the subject based on the results of detection by the plurality of sensors or the angles of joints in any coordinate system based on one of the result of detection by the plurality of sensors.

8. The motion state monitoring system according to claim 1, further comprising the plurality of sensors.

9. A method for controlling a motion state monitoring system configured to monitor a motion of a subject based on a result of detection by each of a plurality of sensors attached to a plurality of respective body parts of a body of the subject, the method comprising:
displaying, on one screen, a measurement result icon indicating a result of measurement of a motion to be monitored and a display setting area for setting an area that displays the result of measurement, the result of measurement being acquirable from the sensor attached to the body part of the body of the subject; and
accepting a setting operation for the display setting area for setting an area that displays the result of measurement for the measurement result icon.

10. A non-transitory computer readable medium storing a control program for causing a computer to execute control processing in a motion state monitoring system configured to monitor a motion of a subject based on a result of detection by each of a plurality of sensors attached to a plurality of respective body parts of a body of the subject, the control processing comprising:
displaying, on one screen, a measurement result icon indicating a result of measurement of a motion to be monitored and a display setting area for setting an area that displays the result of measurement, the result of measurement being acquirable from the sensor attached to the body part of the body of the subject; and
accepting a setting operation for the display setting area for setting an area that displays the result of measurement for the measurement result icon.
